(19) United States
(12) Patent Application Publication
(10) Pub. No.: US 2024/0272132 A1
(43) Pub. Date: Aug. 15, 2024

(54) SENSING DEVICE AND SENSING METHOD

(71) Applicant: Nuvoton Technology Corporation, Hsinchu Science Park (TW)

(72) Inventor: Ming-Chih TSAI, Taichung City (CN)

(21) Appl. No.: 18/441,661

(22) Filed: Feb. 14, 2024

(30) Foreign Application Priority Data
Feb. 14, 2023 (TW) 112105129

(51) Int. Cl. G01N 33/00

(52) U.S. Cl. CPC G01N 33/0027

(57) ABSTRACT

A sensing device and a sensing method are provided. The sensing device includes a substrate, a first unit, a second unit, a third unit, and a fourth unit. The first unit and the second unit are disposed on the substrate and connected to each other in series. The third unit and the fourth unit are disposed on the substrate and connected to each other in series. Of the first unit, the second unit, the third unit, and the fourth unit, two are reference resistors, and the other two are a first sensing unit and a second sensing unit configured to capture volatile organic compounds. At least one of the first sensing unit and the second sensing unit has different capture degrees for polar gas and nonpolar gas of the volatile organic compounds and/or has different capture degrees for protic gas and aprotic gas of the volatile organic compounds.

### CROSS REFERENCE TO RELATED APPLICATIONS

This application claims priority of Taiwan Patent Application No. 112105129, filed on Feb. 14, 2023, the entirety of which is incorporated by reference herein.

### BACKGROUND OF THE INVENTION

### Field of the Invention

The present disclosure relates to sensing device and a sensing method and, in particular, to a sensing device and a sensing method capable of identifying volatile organic compounds.

### Description of the Related Art

With the increasing emphasis on health and safety, various sensing devices are being employed to monitor the pollution levels in the environment. For example, a gas sensor can be used to monitor whether there are harmful gases such as volatile organic compounds (VOCs) in a home environment, an office environment, or a car.

However, although existing sensing devices can sense the presence of volatile organic compounds, they still cannot identify the specific types of volatile organic compounds (such as alcohols, ketones, and benzenes), and as a result their use in various environments and applications is not very widespread. While existing sensing devices and sensing methods have generally met their intended purposes, they are not entirely satisfactory in every respect. Therefore, there are still some issues to be solved with sensing devices and sensing methods.

### BRIEF SUMMARY OF THE INVENTION

A sensing device is provided. The sensing device includes a substrate, a first unit, a second unit, a third unit, and a fourth unit. The first unit and the second unit are disposed on the substrate and connected to each other in series. The third unit and the fourth unit are disposed on the substrate and connected to each other in series. Of the first unit, the second unit, the third unit, and the fourth unit, two are reference resistors, and the other two are a first sensing unit and a second sensing unit configured to capture volatile organic compounds. At least one of the first sensing unit and the second sensing unit has different capture degrees for polar gas and nonpolar gas of the volatile organic compounds, and/or at least one of the first sensing unit and the second sensing unit has different capture degrees for protic gas and aprotic gas of the volatile organic compounds.

A sensing method is provided. The sensing method includes the following steps. A sensing device is provided, wherein the sensing device includes a substrate, a first sensing unit, a second sensing unit, and two reference resistors. The first sensing unit, the second sensing unit, and the two reference resistors are disposed on the substrate. Of the first sensing unit, the second sensing unit, and the two reference resistors, two are connected in series, and the other two are connected in series. Volatile organic compounds are captured by the first sensing unit. The first sensing unit has different capture degrees for polar gas and nonpolar gas of the volatile organic compounds, and/or the first sensing unit has different capture degrees for protic gas and aprotic gas of the volatile organic compounds. The volatile organic compounds are captured by the second sensing unit. The index of gas compound (Indexgas) of the volatile organic compounds is determined by the voltage change in the sensing device.

The sensing device and sensing method disclosed herein can be applied in various types of electronic devices. In order to make the features and advantages of the present disclosure more comprehensible, various embodiments are specially cited below, together with the accompanying drawings, to be described in detail as follows. In order to make the features or advantages of the present disclosure more comprehensible, some embodiments are illustrated herein, and detailed descriptions are provided with reference to the drawings.

### BRIEF DESCRIPTION OF THE DRAWINGS

Aspects of the present disclosure are best understood from the following detailed description when read with the accompanying figures. It should be noted that, in accordance with the standard practice in the industry, various features are not drawn to scale. In fact, the dimensions of the various features may be arbitrarily increased or reduced for clarity of discussion.

FIG. 1 is a schematic diagram of the sensing device according to some embodiments of the present disclosure.

FIG. 2 is a schematic cross-sectional view along line A-A in FIG. 1 according to some embodiments of the present disclosure.

FIG. 3 is a schematic cross-sectional view along line A-A in FIG. 1 according to other embodiments of the present disclosure.

FIG. 4 is a schematic cross-sectional view along line A-A in FIG. 1 according to other embodiments of the present disclosure.

FIG. 5 is a schematic cross-sectional view along line A-A in FIG. 1 according to other embodiments of the present disclosure.

FIG. 6 is a schematic cross-sectional view along line A-A in FIG. 1 according to other embodiments of the present disclosure.

FIG. 7 is a schematic diagram of the sensing unit according to some embodiments of the present disclosure.

FIG. 8 is a schematic diagram of the material particles in the sensing layer according to some embodiments of the present disclosure.

FIG. 9 is a schematic diagram of the sensing device according to other embodiments of the present disclosure.

FIG. 10 is a schematic diagram of the sensing device according to other embodiments of the present disclosure.

FIG. 11 is a function graph of the index of gas compound (Indexgas) and the gas concentration according to some embodiments of the present disclosure.

FIG. 12 is a flowchart of the sensing method according to some embodiments of the present disclosure.

FIG. 13 is a schematic diagram of the index of volatile organic compound (Index_{voc}) according to some embodiments of the present disclosure.

### DETAILED DESCRIPTION OF THE INVENTION

The following disclosure provides many different embodiments, or examples, for implementing different features of the provided neutron beam source generation system, neutron beam source stabilization control system, and neutron beam source generation method. Specific examples of features and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In some embodiments of the present disclosure, terms about disposing and connecting, such as “disposing”, “connecting” and similar terms, unless otherwise specified, may refer to two features are in direct contact with each other, or may also refer to two features are not in direct contact with each other, wherein there is an additional connect feature between the two features. The terms about disposing and connecting may also include the case where both features are movable, or both features are fixed. In addition, the terms “electrically connected” or “electrically coupled” include any direct and indirect means of electrical connection.

In addition, ordinal numbers such as “first”, “second”, and the like used in the specification and claims are configured to modify different features or to distinguish different embodiments or ranges, rather than to limit the number, the upper or lower limits of features, and are not intended to limit the order of manufacture or arrangement of features.

The terms “about”, “substantially”, or the like used herein generally means within 10%, within 5%, within 3%, within 2%, within 1%, or within 0.5% of a given value or a given range. The value given herein is an approximate value, that is, the meaning of “about” may still be implied without the specific description of “about”.

Some variations of the embodiments are described below. In different figures and described embodiments, the same or similar reference numerals are configured to refer to the same or similar features. It should be understood that additional steps may be provided before, during, and after the method, and that some described steps may be replaced or deleted for other embodiments of the method. It should be understood that, in the following embodiments without departing from the spirit of the present disclosure, features in different embodiments may be replaced, reorganized, and combined to form other embodiments. As long as the features in the various embodiments do not violate the spirit of the invention or conflict, they may be used in any combination.

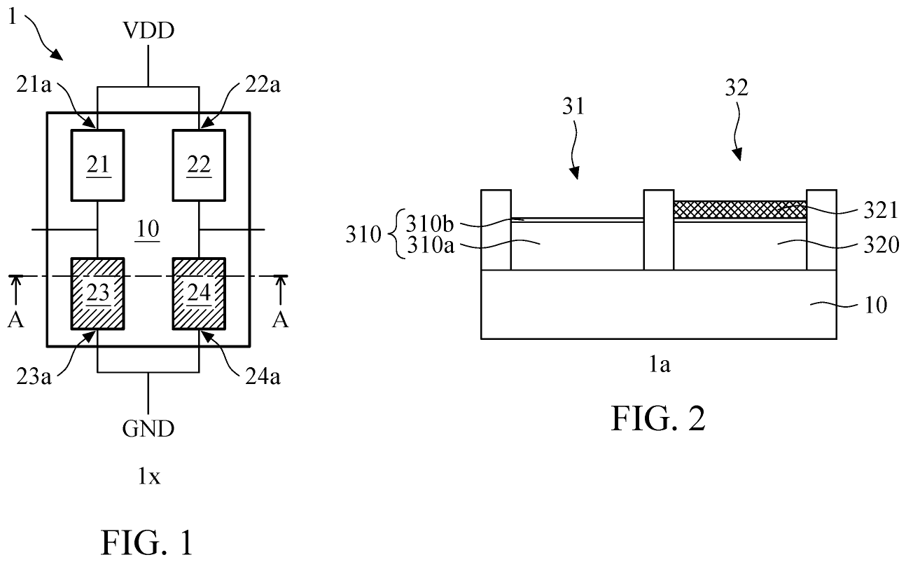

Referring to FIG. 1 and FIG. 2, which are respectively a schematic diagram of the sensing device and a cross-sectional schematic diagram along line A-A in FIG. 1 according to some embodiments of the present disclosure. In some embodiments, the sensing device 1 includes a substrate 10, a first unit 21, a second unit 22, a third unit 23, and a fourth unit 24. In some embodiments, the first unit 21, the second unit 22, the third unit 23, and the fourth unit 24 are adjacently disposed on the substrate 10.

The substrate 10 is configured to carry components (e.g., the first unit 21) thereon. In some embodiments, the substrate 10 may be or include glass, quartz, sapphire, semiconductor, polyimide (PI), polyethylene terephthalate (PET), polycarbonate (PC), polypropylene (PP), rubber, glass fiber, ceramic, other suitable materials, or combinations thereof, but the present disclosure is not limited thereto.

The first unit 21, the second unit 22, the third unit 23, and the fourth unit 24 are configured to provide resistance. In some embodiments, the first unit 21 and the third unit 23 are connected in series, and the second unit 22 and the fourth unit 24 are connected in series. In some embodiments, the terminal 21a of the first unit 21 and the terminal 22a of the second unit 22 are connected in parallel, and the terminals 21a and 22a are electrically coupled to the operating voltage (VDD). In some embodiments, the terminal 23a of the third unit 23 and the terminal 24a of the fourth unit 24 are connected in parallel, and the terminals 23a and 24a are electrically coupled to the ground voltage (GND). In some embodiments, the sensing device 1 has a structure 1x. In the structure 1x, the first unit 21 and the second unit 22 are reference resistors, and the third unit 23 and the fourth unit 24 respectively are a first sensing unit 31 and a second sensing unit 32 configured to capture volatile organic compounds. However, the present disclosure is not limited thereto. In other embodiments, of the first unit 21, the second unit 22, the third unit 23, and the fourth unit 24, any two may be configured as a reference resistor, and the other two may be configured as the first sensing unit 31 and the second sensing unit 32. Other embodiments with different configurations are described below.

In some embodiments, at least one of the first sensing unit 31 and the second sensing unit 32 has different capture degrees for polar gas and nonpolar gas of the volatile organic compounds; and/or at least one of the first sensing unit 31 and the second sensing unit 32 has different capture degrees for protic gas and aprotic gas of the volatile organic compounds. The term “capture” herein refers to the process by which an element (or feature) absorbs gas. For example, when it is described herein that the first sensing unit 31 captures the acetone gas, this means that the first sensing unit 31 absorbs or catches the acetone gas through the specific groups. In addition, the term “capture degree” refers to the affinity of the feature for a gas to be sensed. For example, when it is described herein that the first sensing unit 31 has a higher capture degree for nonpolar gas than the second sensing unit 32, it may refer to the amount (e.g., mole) of the adsorbed nonpolar gas per unit area (e.g., mm²) of the first sensing unit 31 is greater than the amount of the adsorbed nonpolar gas per unit area of the second sensing unit 32. In some embodiments, the term “capture degree” herein may also be replaced with the term “selectivity”. For example, when it is described herein that the selectivity of the first sensing unit 31 for nonpolar gas is larger than that of the second sensing unit 32, it means that the nonpolar gas is more easily captured by the first sensing unit 31.

As shown in FIG. 2, in some embodiments, the sensing device has a structure 1a. In the structure 1a, the first sensing unit 31 (e.g., it may be the third unit 23 in FIG. 1) includes a first sensing layer 310, and the first sensing layer 310 is configured to capture the volatile organic compounds, and the exhibited electrical resistance thereof is changed according to the trapped amount (i.e., adsorbed amount) of the VOCs. For example, the first sensing layer 310 without gas adsorption has an initial resistance, and the first sensing layer 310 with gas adsorption has a measured resistance, wherein the initial resistance is different from the measured resistance. In some embodiments, the first sensing layer 310 may include a carbon substrate 310a and a metal oxide bronze (MOB) 310b, and the metal oxide bronze 310b is disposed over the carbon substrate 310a. In some embodiments, the carbon substrate 310a has a sheet structure, and the metal oxide bronze 310b may be disposed on the upper surface of the carbon substrate 310a. Alternatively, the metal oxide bronze 310b may also cover all outer surfaces of the carbon substrate 310a exposed to the air, rather than the upper surface. In some embodiments, the carbon substrate 310a may be or may include carbon nanoparticles, other suitable carbon materials, or combinations thereof, but the present disclosure is not limited thereto. The metal oxide bronze 310b may be or include titanium metal oxide bronze (Ti-MOB), other suitable metal oxide bronzes, or combinations thereof, but the present disclosure is not limited thereto. The metal oxide bronze 310b is a highly active material, which may be configured to adsorb gas and provide the carbon substrate 310a with the ability to resist the environment. Therefore, the first sensing layer 310 may have an excellent capture degree.

In some embodiments, a capture layer (not shown) may be disposed over the metal oxide bronze 310b in the first sensing layer 310. The capture layer includes a first material having an acetyloxy group (—OAc), and optionally includes a second material having a hydroxyl group (—OH), to improve the sensitivity of the sensing layer 310 to the volatile organic compounds. For example, the first material may be the same as or different from the second material. In embodiments where the first material and the second material are different, the first material and the second material may be in the same layer or may be stacked on top of each other. In some embodiments, the first material may be polyvinyl acetate, poly(vinyl butyral-co-vinyl alcohol-co-vinyl acetate), poly(ethylene-co-vinyl acetate), poly(l-vinylpyrrolidone-co-vinyl acetate), other suitable materials, or combinations thereof, but the present disclosure is not limited thereto. In some embodiments, the second material may be polyvinyl alcohol, poly(vinyl alcohol-co-ethylene), polyvinyl alcohol-polyethylene glycol graft-copolymer), poly(4-vinylphenol), other suitable materials, or combinations thereof, but the present disclosure is not limited thereto. In some embodiments, the acetyloxy group (—OAc) has better affinity for both polar gas and nonpolar gas. In some embodiments, the hydroxyl group (—OH) has better affinity for polar gas, but relatively less affinity for nonpolar gas. By adjusting the ratio of the acetyloxy group (—OAc) to the hydroxyl group (—OH), the effect of capturing specific gases may be effectively realized.

In some embodiments, the molar ratio of acetyloxy group (—OAc):hydroxyl (—OH) in the material (or combination of materials) may be 100˜90:0˜10, but the present disclosure is not limited thereto. For example, the molar ratio of acetyloxy group (—OAc):hydroxyl (—OH) may be 100:0, 98:2, 96:4, 94:6, 92:8, or 90:10, but the present disclosure is not limited thereto. In some embodiments, the molar ratio of acetyloxy group (—OAc):hydroxyl (—OH) in the material may be 100:0, but the present disclosure is not limited thereto.

In some embodiments, the second sensing unit 32 (e.g., the fourth unit 24 in FIG. 1) includes a second sensing layer 320. In some embodiments, the material of the second sensing layer 320 may be similar to or the same as the material of the first sensing layer 310. In other words, the capture degree (gas affinity) of the second sensing layer 320 may be similar to or the same as the capture degree (gas affinity) of the first sensing layer 310.

In some embodiments, the second sensing unit 32 further includes a polar gas selective layer 321 disposed on the second sensing layer 320, and the polar gas selective layer 321 has different degrees for polar gas and nonpolar gas of the volatile organic compounds. In some embodiments, the polar gas selective layer 321 may be or may include a third material having the acetyloxy group (—OAc) and a fourth material having the hydroxide group (—OH). For example, the third material may be the same as or different from the fourth material. In embodiments where the third material is different from the fourth material, the third material and the fourth material may be in the same layer or may be stacked on top of each other. In some embodiments, the third material may be the same as the first material, or may be a combination of the first material with polyvinyl butyral, poly(vinyl cinnamate), poly(vinyl methyl ketone), poly(4-vinylphenol), polyvinylpyrrolidone, other suitable materials, or combinations thereof, but the present disclosure is not limited thereto. In some embodiments, the fourth material may be the same as the second material, or a combination of the second material with polyethylene glycol, polyvinylpyrrolidone, other suitable materials, or combinations thereof, but the present disclosure is not limited thereto.

In some embodiments, the molar ratio of acetyloxy group (—OAc):hydroxyl (—OH) in the polar gas selective layer 321 is 100˜10:0˜90. For example, the molar ratio of acetyloxy group (—OAc):hydroxyl (—OH) may be 100:0, 90:10, 80:20, 70:30, 60:40, 50:50, 40:60, 30:70, 20:80, or 10:90, but the present disclosure is not limited thereto. In some embodiments, the molar ratio of acetyloxy group (OAc):hydroxyl (—OH) in the polar gas selective layer 321 may be 100:0 or 12:88, but the present disclosure is not limited thereto. It should be understood that the types and ratios of the above materials are just examples, but the present disclosure is not limited thereto. In other embodiments, materials with different affinities for polar gas and nonpolar gas may be used in the present disclosure.

In some embodiments, the polar gas selective layer 321 has a greater affinity for polar gas than for nonpolar gas. In other words, the second sensing layer 320 with the polar gas selective layer 321 tends to adsorb polar gases rather than nonpolar gases. In this way, the capture degree of the first sensing unit 31 for nonpolar gas is greater than the capture degree of the second sensing unit 32 for nonpolar gas.

Referring to FIG. 1, in some embodiments, units serving as a reference resistor (e.g., the first unit 21 and the second unit 22) may include a sensing layer and a shielding layer disposed on the sensing layer. For example, the sensing layer of each unit (e.g., the first unit 21 to the fourth unit 24) may be formed in the same process, and the shielding layer is further provided on the sensing layer of the unit serving as a reference resistor, so as to making it unable to absorb gases. In some embodiments, the shielding layer may be or include organic materials, inorganic materials, other suitable materials, or combinations thereof, but the present disclosure is not limited thereto. In some embodiments, the shielding layer may be or include benzocyclobutene, epoxy, polyimide, oxide such as silicon oxide (SiOₓ), nitride such as silicon nitride (SiNₓ), oxynitride such as silicon oxynitride (SiOₓN_{y}), other suitable materials, or combinations thereof, but the present disclosure is not limited thereto. In other embodiments, the unit serving as a reference resistor may also be formed separately, and the material of the unit serving as a reference resistor may be different from the material of the unit serving as a sensing unit.

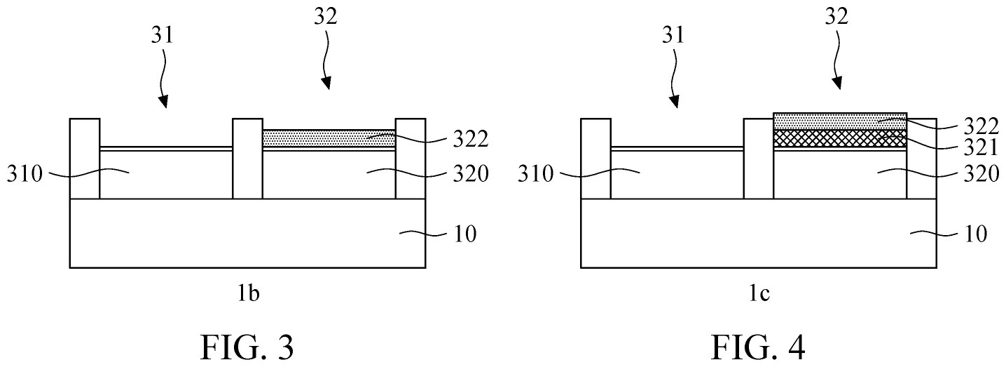

Referring to FIG. 3, which is a schematic cross-sectional view along line A-A in FIG. 1 according to other embodiments of the present disclosure. In some embodiments, the sensing device has structure 1b. In the structure 1b, the first sensing unit 31 includes a first sensing layer 310, and the second sensing unit 32 includes a second sensing layer 320 and a protic gas selective layer 322 disposed on the second sensing layer 320. Moreover, the protic gas selective layer 322 has different capture degrees for protic gas and aprotic gas of the volatile organic compounds.

In some embodiments, the protic gas selective layer 322 may be or may include a fifth material having the acetyloxy group (—OAc) and a sixth material having the hydroxyl group (—OH). For example, the fifth material may be the same as or different from the sixth material. In an embodiment where the fifth material is different from the sixth material, the fifth material and the sixth material may be in the same layer, or may be stacked on top of each other. In some embodiments, the fifth material may be the same as the first material, or may be a combination of the first material with polyvinyl butyral, poly(vinyl cinnamate), poly(vinyl methyl ketone), poly(4-vinylphenol), polyvinylpyrrolidone, other suitable materials, or combinations thereof, but the present disclosure does not limited thereto. In some embodiments, the sixth material may be the same as the second material, or may be a combination of the second material with polyethylene glycol, polyvinylpyrrolidone, other suitable materials, or combinations thereof, but the present disclosure is not limited thereto.

In some embodiments, the molar ratio of acetyloxy group (—OAc):hydroxyl (—OH) in the protic gas selective layer 322 is 0˜20:100˜80. For example, the molar ratio of acetyloxy group (—OAc):hydroxyl (—OH) may be 0:100, 2:98, 4:96, 6:94, 8:92, 10:90, 12:88, 14:86, 16:84, 18:82, or 20:80. In some embodiments, the molar ratio of acetyloxy group (—OAc):hydroxyl (—OH) in the protic gas selective layer 322 may be 12:88 or 2:98, but the present disclosure is not limited thereto. It should be understood that the types and ratios of the above materials are just examples, but the present disclosure is not limited thereto. In other embodiments, materials with different affinities for protic gas and aprotic gas may be used in the present disclosure.

In some embodiments, the protic gas selective layer 322 has a greater affinity for protic gas than for aprotic gas. In other words, the second sensing layer 320 with the protic gas selective layer 322 tends to adsorb protic gases rather than aprotic gases. In this way, the capture degree of the first sensing unit 31 for aprotic gas is greater than the capture degree of the second sensing unit 32 for aprotic gas.

Referring to FIG. 4, which is a schematic cross-sectional view along line A-A in FIG. 1 according to other embodiments of the present disclosure. In some embodiments, the sensing device has structure 1c. In the structure 1c, the first sensing unit 31 includes a first sensing layer 310, and the second sensing unit 32 includes a second sensing layer 320, a polar gas selective layer 321 disposed on the second sensing layer 320, and a protic gas selective layer 322 disposed on the polar gas selective layer 321. In some embodiments, the capture degree of the first sensing unit 31 for nonpolar gas is greater than the capture degree of the second sensing unit 32 for nonpolar gas, and the capture degree of the first sensing unit 31 for aprotic gas is greater than the capture degree of the second sensing unit 32 for aprotic gas. It should be noted that although FIG. 4 shows that the protic gas selective layer 322 is disposed over the polar gas selective layer 321, the present disclosure is not limited thereto. In other embodiments, the polar gas selective layer 321 may also be disposed over the protic gas selective layer 322, and the protic gas selective layer 322 is located between the second sensing layer 320 and the polar gas selective layer 321.

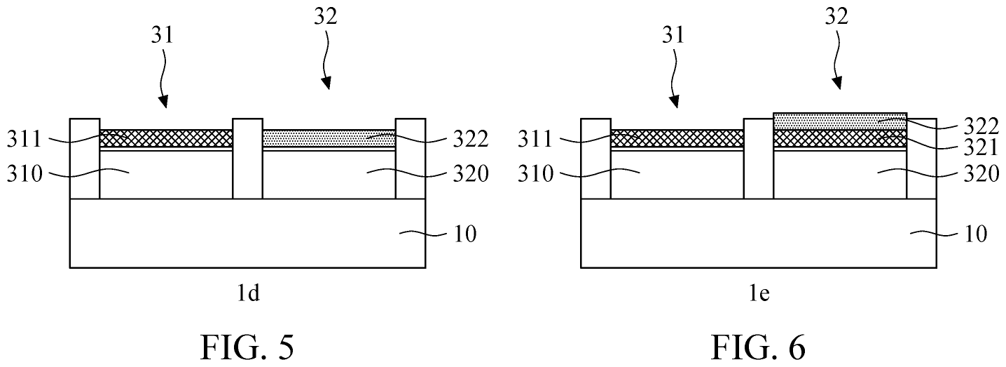

Referring to FIG. 5, which is a schematic cross-sectional view along line A-A in FIG. 1 according to other embodiments of the present disclosure. In some embodiments, the sensing device has structure 1d. In the structure 1d, the first sensing unit 31 includes a first sensing layer 310 and a polar gas selective layer 311 disposed on the first sensing layer 310, and the second sensing unit 32 includes a second sensing layer 320 and a protic gas selective layer 322 on the second sensing layer 320.

In some embodiments, the material of the polar gas selective layer 311 may be similar to or the same as the material of the polar gas selective layer 321, so the described is omitted. In some embodiments, the capture degree of the first sensing unit 31 for nonpolar gas is smaller than the capture degree of the second sensing unit 32 for nonpolar gas, and the capture degree of the first sensing unit 31 for aprotic gas is greater than the capture degree of the second sensing unit 31 for aprotic gas.

Referring to FIG. 6, which is a schematic cross-sectional view along line A-A in FIG. 1 according to some other embodiments of the present disclosure. In some embodiments, the sensing device has structure 1e. In the structure 1e, the first sensing unit 31 includes a first sensing layer 310 and a polar gas selective layer 311 disposed on the first sensing layer, and the second sensing unit 32 includes a second sensing layer 320, a polar gas selective layer 321 on the second sensing layer 320, and a protic gas selective layer 322 disposed on the polar gas selective layer 321. In some embodiments, the material of the polar gas selective layer 311 may be similar to or the same as the material of the polar gas selective layer 321. In some embodiments, the capture degree of the first sensing unit 31 for aprotic gas is greater than the capture degree of the second sensing unit 32 for aprotic gas.

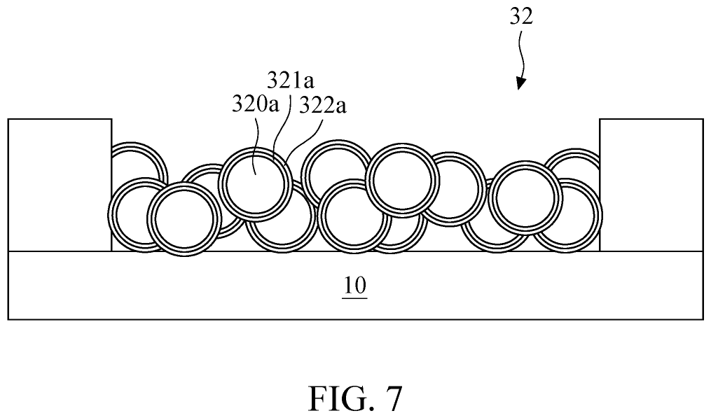

It should be noted that although the sensing units shown in FIGS. 2 to 6 are stacks of multiple film layers, the present disclosure is not limited thereto. In some embodiments, the first sensing layer 310 and the second sensing layer 320 may respectively have a plurality of sensing particles, and the polar gas selective layer and the protic gas selective layer are disposed to cover the sensing particles. The sensing particles may be nano-carbon particles coated with metal oxide bronze. For example, referring to FIG. 7, which is a schematic diagram of the sensing unit according to some embodiments of the present disclosure. As shown in the figure, in some embodiments, the second sensing unit 32 includes a second sensing layer 320 composed of a plurality of sensing particles 320a, a polar gas selective layer 321a disposed on the sensing particles 320a, and a protic gas selective layer 322a on the polar gas selective layer 321a. It should be noted that the sensing unit shown in FIG. 7 is only an example, and the present disclosure is not limited thereto. In some embodiments, the protic gas selective layer 322a may be disposed over the sensing particle 320a, and then the polar gas selective layer 321a may be disposed over the protic gas selective layer 322a. In some embodiments, any one or both of the polar gas selective layer and the protic gas selective layer in the sensing unit may be omitted.

By disposing different selective layers (that is, the polar gas selective layer and the protic gas selective layer), the capture degree of the first sensing unit 31 and the second sensing unit 32 for different types of gases may be adjusted, so that the first sensing unit 31 and the second sensing unit 32 exhibit different measurement resistances (or measurement voltages). An index of gas compound (Indexgas) of the volatile organic compounds may be further determined by the change of resistance (or voltage). The index of gas compound (Indexgas) may be converted into a gas concentration (e.g., ppm).

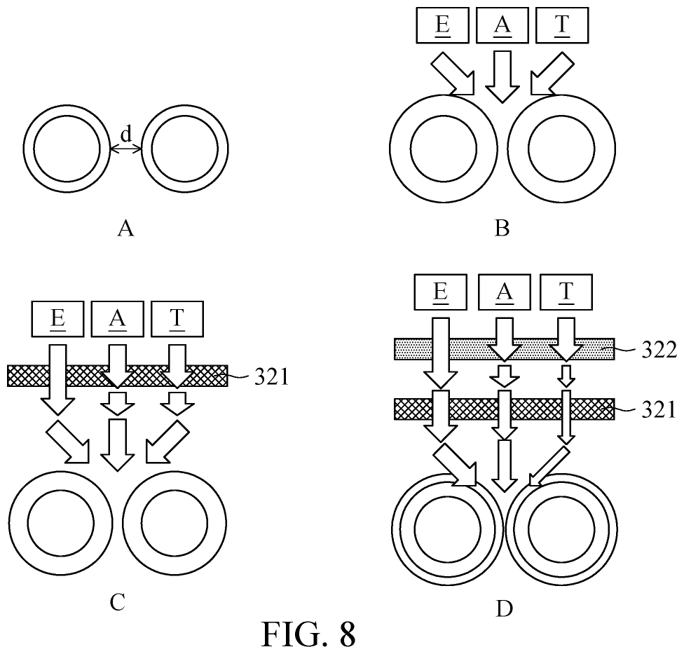

In order to make the present disclosure clearer and easier to understand, referring to FIG. 8, which is a schematic diagram of the material particles in the sensing layer according to some embodiments of the present disclosure. In some embodiments, the resistance may be regarded as the sum of an intrinsic resistance (R_{resistivity}) and a steric resistance (R_{steric}). Specifically, the intrinsic resistance (R_{resistivity}) is related to the nature of the material particles, and the steric resistance (R_{steric}) is related to the spatial spacing of the material particles. As shown in part A of FIG. 8, the material particles in the sensing layer (e.g., the first sensing layer 310 or the second sensing layer 320) are not adsorbed with gas, and there is a distance d between adjacent material particles. Therefore, the material particles have an initial resistance of R. At this time, the resistance of the material particles in part A is R, and R=R_{resistivity}+R_{steric}.

As shown in Part B of FIG. 8, when the material particles are adsorbed with the volatile organic compounds such as ethanol (E), acetone (A) and toluene (T), the distance d between adjacent material particles changes. Affected by that the distance d changes, the resistance of the material particles also changes. Specifically, compared with the state where no gas is adsorbed, the intrinsic resistance (R_{resistivity}) of the material particles remains constant, but the steric resistance (R_{steric}) increases due to the increase in the distance d between adjacent material particles. At this time, the difference between the resistance of the material particles in part B and the resistance of the material particles in part A (i.e., the resistance of part B minus the resistance of part A) is AR, and the resistance of the material particles in part B is R+ΔR.

As shown in part C of FIG. 8, the polar gas selective layer 321 is disposed on the material particles. Since the polar gas selective layer 321 tends to capture polar gases, the amount of nonpolar gases adsorbed by the material particles decreases. For example, the amount of toluene adsorbed by the material particles in Part C is less than the amount of toluene adsorbed by the material particles in Part B. Therefore, compared with the portion B, the change in the distance d of portion C is less than the change in the distance d of portion B, and the change in the resistance of portion C is less than the change in the resistance of portion B. Specifically, compared with the state where no gas is adsorbed, the intrinsic resistance (R_{resistivity}) of the material particles remains constant, but the steric resistance (R_{steric}) increases slightly because the distance d between adjacent material particles only slightly increases. At this time, the difference between the resistance of the material particles of part C and the resistance of the material particles of part B (i.e., the resistance of part C minus the resistance of part B) is −ΔR′, and the resistance of the material particles of part C is R+ΔR−ΔR′.

As shown in part D of FIG. 8, a protic gas selective layer 322 is further disposed on the material particles. Since the protic gas selective layer 322 tends to capture protic gases, the amount of aprotic gases adsorbed by the material particles decrease. For example, the amount of acetone adsorbed by the material particles in Part D is less than the amount of acetone adsorbed by the material particles in Part C. Therefore, compared with part C, the change in the distance d of portion D is less than the change in the distance d of portion C, and the change in the resistance of portion D is less than the change in the resistance of portion C. Specifically, compared with the state where no gas is adsorbed, the intrinsic resistance (R_{resistivity}) of the material particles remains constant, but the steric resistance (R_{steric}) increases slightly because the distance d between adjacent material particles only slightly increases. At this time, the difference between the resistance of the material particles of part D and the resistance of the material particles of part C (the resistance of part D minus the resistance of part C) is −ΔR″, and the resistance of the material particles of part D is R+ΔR−ΔR′−ΔR″.

According to the above, when the first sensing unit 31 and the second sensing unit 32 are provided with a polar gas selective layer and/or a protic gas selective layer, the values of R, ΔR, ΔR′, and ΔR″ may be determined by cross-comparison of the resistances of the first sensing unit 31 and the second sensing unit 32. In addition, by using the R, ΔR, ΔR′ and ΔR″, the index of gas compound (Index_{gas}) may be calculated, and the index of gas compound (Index_{gas}) may be converted into a gas concentration. Some embodiments according to the present disclosure will be provided as illustrations below.

In the following, V_{O} is the measurement voltage, VDD is the operating voltage, R is the initial resistance, ΔR is the resistance change, ΔR′ is the resistance change related to the polar gas selective layer, ΔR″ is the resistance change related to the protic gas selective layer.

In some embodiments, the sensing device is configured as structure 1x as shown in FIG. 1. In this case, when the first sensing unit 31 and the second sensing unit 32 have the structure 1a shown in FIG. 2, the resistance of the first unit 21 is R, the resistance of the second unit 22 is R, the resistance of the third unit 23 is R+ΔR, and the resistance of the fourth unit 24 is R+ΔR−ΔR′. Therefore, the relationship between voltage and resistance may be expressed as

$$Vo = \frac{R * Δ {R}^{′}}{( 2 R + Δ R ) ( 2 R + Δ R - Δ {R}^{′} )} \times VDD .$$

Alternatively, when the first sensing unit 31 and the second sensing unit 32 have the structure 1c shown in FIG. 4, the resistance of the first unit 21 is R, the resistance of the second unit 22 is R, the resistance of the third unit 23 is R+ΔR, and the resistance of the fourth unit 24 is R+ΔR−ΔR′-ΔR″. Therefore, the relationship between voltage and resistance may be expressed as

$$Vo = \frac{R * ( Δ {R}^{′} + Δ {R}^{″} )}{( 2 R + Δ R ) ( 2 R + Δ R - Δ {R}^{′} - Δ {R}^{″} )} \times VDD .$$

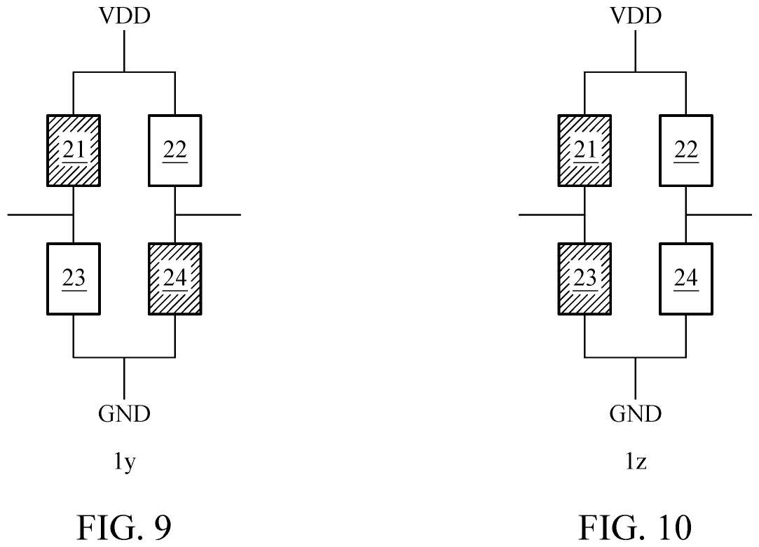

Referring to FIG. 9, which is a schematic diagram of the sensing device according to other embodiments of the present disclosure. In some embodiments, the sensing device is configured as structure 1y as shown in FIG. 9. Specifically, the second unit 22 and the third unit 23 are reference resistors, and the first unit 21 and the fourth unit 24 are respectively the first sensing unit 31 and the second sensing unit 32. In this case, when the first sensing unit 31 and the second sensing unit 32 have the structure 1a shown in FIG. 2, the resistance of the first unit 21 is R+ΔR, the resistance of the second unit 22 is R, the resistance of the third unit 23 is R, and the resistance of the fourth unit 24 is R+ΔR−ΔR′. Therefore, the relationship between voltage and resistance may be expressed as

$$Vo = \frac{- Δ R ( 2 R + Δ R ) + Δ {R}^{′} ( R + Δ R )}{( 2 R + Δ R ) ( 2 R + Δ R - Δ {R}^{′} )} \times VDD .$$

Alternatively, when the first sensing unit 31 and the second sensing unit 32 have the structure 1c shown in FIG. 4, the resistance of the first unit 21 is R+ΔR, the resistance of the second unit 22 is R, the resistance of the third unit 23 is R, and the resistance of the fourth unit 24 is R+ΔR−ΔR′−ΔR″. Therefore, the relationship between voltage and resistance may be expressed as

$$Vo = [ \frac{- Δ R ( 2 R + Δ R - Δ {R}^{′} - Δ {R}^{″} ) + R ( Δ {R}^{′} + Δ {R}^{″} )}{( 2 R + Δ R ) ( 2 R + Δ R - Δ {R}^{′} - Δ {R}^{″} )} ] \times VDD .$$

Referring to FIG. 10, which is a schematic diagram of the sensing device according to other embodiments of the present disclosure. In some embodiments, the sensing device is configured as structure 1z as shown in FIG. 10. Specifically, the first unit 21 and the second unit 22 are reference resistors, and the third unit 23 and the fourth unit 24 are respectively the first sensing unit 31 and the second sensing unit 32. In this case, when the first sensing unit 31 and the second sensing unit 32 have the structure 1a shown in FIG. 2, the resistance of the first unit 21 is R+ΔR, the resistance of the second unit 22 is R, the resistance of the third unit 23 is R+ΔR−ΔR′, and the resistance of the fourth unit 24 is R. Therefore, the relationship between voltage and resistance may be expressed as

$$Vo = \frac{- Δ {R}^{′}}{2 ( 2 R + 2 Δ R - Δ {R}^{′} )} \times VDD .$$

Alternatively, when the first sensing unit 31 and the second sensing unit 32 have the structure 1c shown in FIG. 4, the resistance of the first unit 21 is R+ΔR, the resistance of the second unit 22 is R, the resistance of the third unit 23 is R+ΔR−ΔR′−ΔR″, and the resistance of the fourth unit 24 is R. Therefore, the relationship between voltage and resistance may be expressed as

$$Vo = \frac{- ( Δ {R}^{′} + Δ {R}^{″} )}{2 ( 2 R + 2 Δ R - Δ {R}^{′} - Δ {R}^{″} )} \times VDD .$$

As mentioned above, after obtaining the values of V_{O}, Δ_{V(Gas-F)2}, ΔV_{(Gas-F)1}, Vₒ₀, R, ΔR, ΔR′, and ΔR″, the index of gas compound (Index_{gas}) may be obtained by the following formula. In some embodiments, the index of gas compound (Index_{gas}) may be expressed as Vₒ×100-Vₒ₀×100. Alternatively, the index of gas compound (Index_{gas}) may also be expressed as (ΔV_{(Gas-F)2}−ΔV_{(Gas-F)1})×100. Specifically, V_{O} is the measured voltage, Vₒ₀ is the initial measured voltage when not in contact with gas, ΔV_{(Gas-F)2} is the voltage change of the second sensing unit 32, and ΔV_{(Gas-F)1} is the voltage change of the first sensing unit 31.

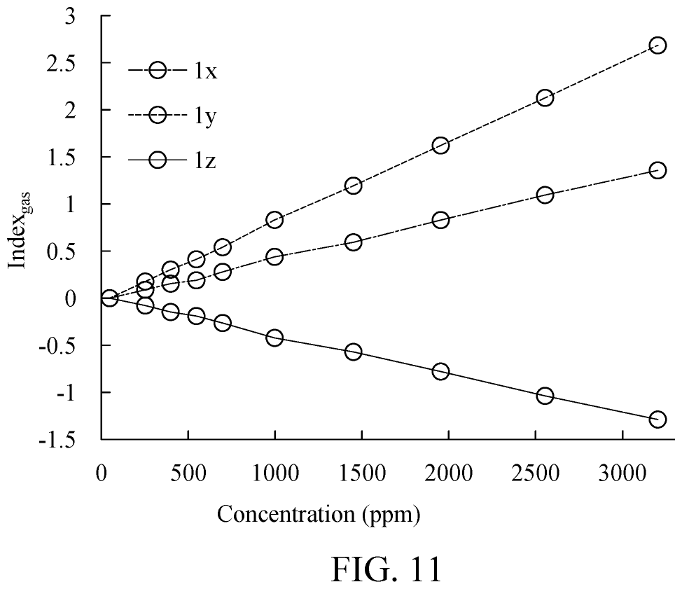

Referring to FIG. 11, which is a function graph of the index of gas compound (Index_{gas}) and gas concentration according to some embodiments of the present disclosure. In some embodiments, the gas concentration may be obtained by the function graph in FIG. 11 according to the index of gas compound (Index_{gas}) obtained by various structures (1x˜1c) of the sensing device. For example, when the index of gas compound (Index_{gas}) calculated by the sensing device with the structure 1x is 0.5, the result that the gas concentration is 1500 ppm may be obtained.

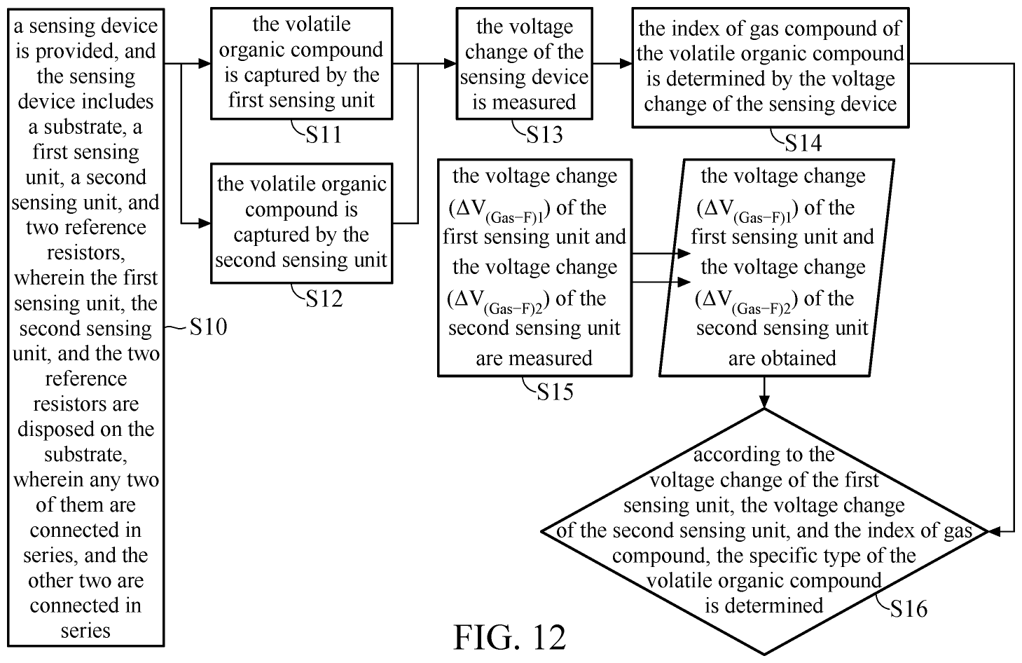

Referring to FIG. 12, which is a flowchart of the sensing method according to some embodiments of the present disclosure. In some embodiments, the sensing method includes steps S10 to S16. In step S10, a sensing device is provided. The sensing device includes a substrate, a first sensing unit, a second sensing unit, and two reference resistors. The first sensing unit, the second sensing unit, and the two reference resistors are disposed on the substrate, wherein any two of them are connected in series, and the other two are connected in series. For example, in the sensing device provided in step S10, the first sensing unit is connected in series with a reference resistor, and the second sensing unit is connected in series with another reference resistor (such as the structure 1x shown in FIG. 1). However, the present disclosure is not limited thereto. In other embodiments, in the sensing device provided in step S10, the first sensing unit and the second sensing unit may also be connected in series, and two reference resistors may be connected in series (such as the structure 1z shown in FIG. 10).

In step S11, the volatile organic compounds are captured by the first sensing unit, wherein the first sensing unit has different capture degrees for polar gas and nonpolar gas of the volatile organic compounds, and/or the first sensing unit has different capture degrees for protic gas and aprotic gas of the volatile organic compounds. For example, the above-mentioned polar gas selective layer and/or the above-mentioned protic gas selective layer may be disposed on the first sensing unit to adjust the capture degree of the first sensing unit for different types of gases. In this case, the physical change (e.g., voltage or resistance) measured by the first sensing unit will be affected by the capture degree of the selective layer for a specific type of gas.

In step S12, the volatile organic compounds are captured by the second sensing unit. For example, by not providing the above-mentioned polar gas selective layer and/or the above-mentioned protic gas selective layer on the second sensing unit, the physical change (for example, voltage or resistance) measured by the second sensing unit is affected by the capture degree of all kinds of gases. In step S13, the voltage change of the sensing device is measured. In step S14, the index of gas compound (Index_{gas}) of the volatile organic compound is determined by the voltage change of the sensing device. For example, the index of gas compound (Index_{gas}) may be determined by the formula described above.

In step S15, the voltage change (ΔV_{(Gas-F)1}) of the first sensing unit and the voltage change (ΔV_{(Gas-F)2}) of the second sensing unit are measured. For example, when the sensing device is configured as the structure 1x shown in FIG. 1, the voltage change (ΔV_{(Gas-F)1}) of the first sensing unit 31 and the voltage change (ΔV_{(Gas-F)2}) of the second sensing unit 32 may be measured directly. However, the present disclosure is not limited thereto. In other embodiments, a plurality of different types of sensing devices may be provided, and the voltage change (ΔV_{(Gas-F)1}) of the first sensing unit 31 and the voltage change (ΔV_{(Gas-F)2}) of the second sensing unit 32 may be obtained by cross-comparison.

In step S16, according to the voltage change (or resistance change) of the first sensing unit, the voltage change (or resistance change) of the second sensing unit, and the index of gas compound (Index_{gas}), the specific type of the volatile organic compound is determined. That is, whether the volatile organic compound is a polar gas or whether the volatile organic compound is a protic gas is determined. In some embodiments, steps S15 and S16 may also be replaced by the following methods to determine the types of volatile organic compounds. Firstly, a first sensing device and a second sensing device are provided, wherein the first sensing unit of the first sensing device includes a polar gas selective layer and a protic gas selective layer, and the second sensing unit does not include a selective layer. The first sensing unit of the second sensing device includes a protic gas selective layer, and the second sensing unit does not include a selective layer. Next, the physical property changes (e.g., resistance change, or voltage change) caused by the volatile organic compounds on the first sensing device and the second sensing device are measured to calculate an index of gas compound (Index_{gas}). Then, a volatile organic compound index (Index_{voc}) is defined based on the index of ethanol gas compound (Index_{gas}). Specifically, the volatile organic compound index (Index_{voc}) is equal to the value of the measured index of gas compound (Index_{gas})/index of ethanol gas compound (Index_{gas})*100. For example, when the measured gas is ethanol, the index of volatile organic compounds (Index_{voc}) is 100.

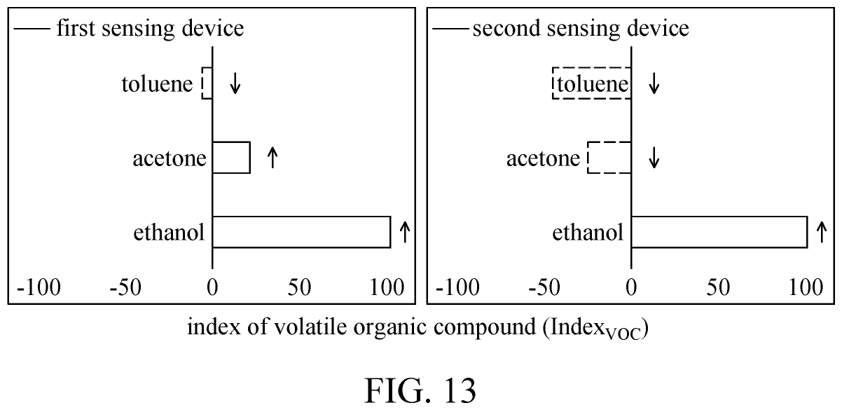

As mentioned above, since the first sensing device includes a polar gas selective layer and a protic gas selective layer, the first sensing device has the highest capture degree for ethanol (which is a polar gas and a protic gas), the second highest degree capture for acetone (which is a polar gas and an aprotic gas), and the lowest capture degree for toluene (which is a nonpolar gas). Taking FIG. 13 as an example, the index of volatile organic compound (Index_{voc}) measured by the first sensing device for ethanol, acetone and toluene respectively are 100, 25, and −10. On the other hand, since the second sensing device only includes a protic gas selective layer, the capture degree of the second sensing device for ethanol (which is a protic gas) is significantly higher than that for acetone (which is an aprotic gas) and the capture degree for toluene. Taking FIG. 13 as an example, the index of volatile organic compound (Index_{voc}) measured by the second sensing device for ethanol, acetone and toluene respectively are 100, −25, and −50.

Therefore, when a volatile organic compound needs to be qualified, the type of the volatile organic compound may be determined by comparing the volatile organic compound index (Index_{voc}) measured by the first sensing device and the second sensing device. For example, when the indexes of volatile organic compound (Index_{voc}) measured by the first sensing device and the second sensing device respectively are a positive value (e.g., +25) and a negative value (e.g., −25), then it may be determined that the volatile organic compound is acetone.

As mentioned above, the present disclosure provides a sensing device and a sensing method that can effectively identify the types of volatile organic compounds, for application in various electronic products.

Components in the disclosed embodiments may be mixed and matched arbitrarily as long as they do not violate the spirit of the invention or conflict with each other. In addition, the scope of protection of the present disclosure is not limited to the process, machine, manufacture, material composition, device, method and steps in the specific embodiments described in the specification, any person with ordinary knowledge in the field may learn from the contents of the present disclosure Understand the process, machine, manufacture, material composition, device, method and steps developed in the present or in the future, as long as they may implement substantially the same function or obtain substantially the same result in the embodiments described herein, they may be used according to the present disclosure. Therefore, the scope of protection of the present disclosure includes the above-mentioned process, machine, manufacture, composition of matter, device, method and steps. Any embodiment or claims of the present disclosure need not achieve all the objectives, advantages and/or features disclosed in the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A sensing device, comprising:
a substrate;
a first unit disposed on the substrate;
a second unit disposed on the substrate and connected in series with the first unit;
a third unit disposed on the substrate; and
a fourth unit disposed on the substrate and connected in series with the third unit,
wherein of the first unit, the second unit, the third unit, and the fourth unit, two are reference resistors, and the other two are a first sensing unit and a second sensing unit configured to capture volatile organic compounds (VOCs), wherein
at least one of the first sensing unit and the second sensing unit has different capture degrees for polar gas and nonpolar gas of the volatile organic compounds; and/or,
at least one of the first sensing unit and the second sensing unit has different capture degrees for protic gas and aprotic gas of the volatile organic compounds.

2. The sensing device as claimed in claim 1, wherein the first sensing unit comprises a first sensing layer, and the second sensing unit comprises a second sensing layer and a polar gas selective layer disposed on the second sensing layer, wherein the polar gas selective layer has different capture degrees for polar gas and nonpolar gas of the volatile organic compounds.

3. The sensing device as claimed in claim 2, wherein a capture degree of the first sensing unit for nonpolar gas is greater than a capture degree of the second sensing unit for nonpolar gas.

4. The sensing device as claimed in claim 1, wherein the first sensing unit comprises a first sensing layer, and the second sensing unit comprises a second sensing layer and a protic gas selective layer disposed on the second sensing layer, wherein the protic gas selective layer has different capture degrees for protic gas and aprotic gas of the volatile organic compounds.

5. The sensing device as claimed in claim 4, wherein a capture degree of the first sensing unit for aprotic gas is greater than a capture degree of the second sensing unit for aprotic gas.

6. The sensing device as claimed in claim 4, wherein the protic gas selective layer has different capture degrees for polar gas and nonpolar gas of the volatile organic compounds.

7. The sensing device as claimed in claim 1, wherein the first sensing unit comprises a first sensing layer, and the second sensing unit comprises a second sensing layer, a polar gas selective layer disposed on the second sensing layer, and a protic gas selective layer disposed on the polar gas selective layer, wherein the polar gas selective layer has different capture degrees for polar gas and nonpolar gas of the volatile organic compounds, and the protic gas selective layer has different capture degrees for protic gas and aprotic gas of the volatile organic compounds.

8. The sensing device as claimed in claim 7, wherein a capture degree of the first sensing unit for nonpolar gas is greater than a capture degree of the second sensing unit for nonpolar gas, and a capture degree of the first sensing unit for aprotic gas is greater than a capture degree of the second sensing unit for aprotic gas.

9. The sensing device as claimed in claim 1, wherein the first sensing unit comprises a first sensing layer and a polar gas selective layer disposed on the first sensing layer, and the polar gas selective layer has different capture degrees for polar gas and nonpolar gas of the volatile organic compounds, wherein the second sensing unit comprises a second sensing layer and a protic gas selective layer disposed on the second sensing layer, and the protic gas selective layer has different capture degrees for protic gas and aprotic gas of the volatile organic compounds.

10. The sensing device as claimed in claim 9, wherein a capture degree of the first sensing unit for nonpolar gas is smaller than a capture degree of the second sensing unit for nonpolar gas, and a capture degree of the first sensing unit for aprotic gas is greater than a capture degree of the second sensing unit for aprotic gas.

11. The sensing device as claimed in claim 1, wherein the first sensing unit comprises a first sensing layer and a polar gas selective layer disposed on the first sensing layer, and the second sensing unit comprises a second sensing layer, the polar gas selective layer disposed on the second sensing layer, and a protic gas selective layer disposed on the polar gas selective layer, wherein the polar gas selective layers have different capture degrees for polar gas and nonpolar gas of the volatile organic compounds, and the protic gas selective layer has different capture degrees for protic gas and aprotic gas of the volatile organic compounds.

12. The sensing device as claimed in claim 11, wherein a capture degree of the first sensing unit for aprotic gas is greater than a capture degree of the second sensing unit for aprotic gas.

13. The sensing device as claimed in claim 11, wherein the first sensing layer and the second sensing layer have a plurality of sensing particles, and the polar gas selective layer and the protic gas selective layer are disposed to cover the plurality of sensing particles.

14. The sensing device as claimed in claim 1, wherein the second unit and the fourth unit are reference resistors, and the first unit and the third unit respectively are the first sensing unit and the second sensing unit.

15. The sensing device as claimed in claim 1, wherein the second unit and the third unit are reference resistors, and the first unit and the fourth unit respectively are the first sensing unit and the second sensing unit.

16. The sensing device as claimed in claim 1, wherein the first unit and the second unit are reference resistors, and the third unit and the fourth unit respectively are the first sensing unit and the second sensing unit.

17. The sensing device as claimed in claim 1, wherein the first sensing unit has different capture degrees for polar gas and nonpolar gas of the volatile organic compounds, and the first sensing unit has a material having an acetyloxy group (—OAc) and a material having a hydroxyl (—OH), wherein a molar ratio of acetyloxy group (—OAc):hydroxyl (—OH) is 100˜10:0˜90.

18. The sensing device as claimed in claim 1, wherein the first sensing unit has different capture degrees for protic gas and aprotic gas of the volatile organic compounds, and the first sensing unit has a material having an acetyloxy group (—OAc) and a material having a hydroxyl (—OH), wherein a molar ratio of acetyloxy group (—OAc):hydroxyl (—OH) is 0˜20:100˜80.

19. A sensing method, comprising:
providing a sensing device, wherein the sensing device comprises a substrate, a first sensing unit, a second sensing unit, and two reference resistors, wherein the first sensing unit, the second sensing unit, and the two reference resistors are disposed on the substrate, wherein of the first sensing unit, the second sensing unit, and the two reference resistors, two are connected in series, and the other two are connected in series;
capturing volatile organic compounds using the first sensing unit, wherein
the first sensing unit has different capture degrees for polar gas and nonpolar gas of the volatile organic compounds, and/or the first sensing unit has different capture degrees for protic gas and aprotic gas of the volatile organic compounds;
capturing the volatile organic compounds using the second sensing unit; and
determining index of gas compound (Index_{gas}) of the volatile organic compounds using voltage change of the sensing device.

20. The sensing method as claimed in claim 19, wherein the first sensing unit is connected in series with one of the two reference resistors, the second sensing unit is connected in series with the other of the two reference resistors, and the sensing method further comprises:
determining gas type of the volatile organic compounds using voltage change of the first sensing unit and the second sensing unit.
